(19) United States
(12) Patent Application Publication
(10) Pub. No.: US 2013/0030529 A1
(43) Pub. Date: Jan. 31, 2013

(54) IMPLANT INTERFACE SYSTEM AND METHOD

(71) Applicant: Jessee Hunt, Plano, TX (US)

(72) Inventor: Jessee Hunt, Plano, TX (US)

(21) Appl. No.: 13/194,561

(22) Filed: Jul. 29, 2011

(51) Int. Cl. A61F 2/28

(52) U.S. Cl. 623/16.11

(57) ABSTRACT

An orthopedic implant having an implant body including a bone interface surface having a bone interface structure protruding therefrom. The bone interface structure includes a proximal portion of the bone interface structure adjacent the bone interface surface and a distal portion of the bone interface structure extending from the proximal portion of the bone interface structure, wherein the distal portion of the bone interface structure configured to be disposed at least partially into a bone structure during use.

### BACKGROUND

1. Field of the Invention

The present invention relates generally to medical devices and, more particularly to implants.

2. Description of Related Art

Implants may be used in human and/or animals to support and/or secure one or more bones. Orthopedic implants are designed to be placed in the body as a replacement for damaged joints or repair of broken bones. For example, a knee replacement procedure may include replacing diseased or damaged joint surfaces of the knee with implants, such as metal and plastic components shaped to allow continued motion of the knee. Although orthopedic implants and procedures are common and have improved over the years, current implant designs may be susceptible to drawbacks, such as in insufficient interface between the bone and the implant. The bone-implant interface may significantly impact how an implant integrates into the patient's anatomy and, thus, may directly impact long term success of an implant procedure. Providing a sufficient bone-implant interface may be of increased importance where the implant is subject to loading, such as with knee replacements.

The direct structural and functional connection between living bone and the surface of a load-bearing implant is often referred to as osteointegration. Wolf's Law relating to osteointegration is a recognized theory that bone in a healthy person or animal will adapt to the loads it is placed under. If loading on a particular bone increases, the bone will remodel itself over time to become stronger to resist that sort of loading (the external cortical portion of the bone becomes thicker). The converse is true as well: if the loading on a bone decreases, the bone will become weaker due to turnover, it is less metabolically costly to maintain and there is no stimulus for continued remodeling that is required to maintain bone mass.

Current implant designs use various techniques in an attempt to provide strong initial fixation and long-term fixation. For example, joint replacement implants for the knee, hip, shoulder ankle often include posts or screws that provide initial fixation. . Unfortunately, these fixation techniques often exhibit deficiencies, including varied and inadequate stress distribution throughout the bone-implant interface. Inadequate stress distribution at the bone/implant interface may ultimately lead to a reduction in bone density and thereby cause loosening of the implant. In some instances, implants include a porous coating to promote adhesion to the bone (e.g., by way of bone-ingrowth). Due to multidirectional forces being applied to implants at any given point in time, these coatings may not offer sufficient initial fixation. This lack of fixation may enable micromotion which may lead to irregular bone healing and remodeling, lack of adherence and non-uniformity. Additionally porous coatings may not provide sufficient thickness to facilitate effective bone tissue in-growth within the dynamic environment that implants exist. Such inadequate structural designs often lead to inadequate long term fixation due to issues such as implant component loosening, implant instability, migration of the implant, rotation of the implant, premature wear on articulating surfaces of the bone or implant, periprosthetic fractures of bone at or near the bone-implant interface, as well as other issues.

Further, in procedures that require fixation to a boney structure, the boney structure may have to be prepared to accept the implant. In some instances, a significant amount of bone may be cut away to prepare the bone for the implant, thereby leaving a void that is compensated for by using an implant of an increased size. In the case of a knee implant, for example, weight-bearing surfaces of the knee joint may be removed, with an implant residing in its place. The height of the implant may be increased or decreased to account for the amount of removed bone to avoid differences between the length of the leg having the knee implant and the other leg. Unfortunately, an increase in size of the implant to account for the removed boney structure can lead to added implant complexity, and may still suffer from drawbacks relating to fixation of the implant to the bone structure, as discussed above.

Accordingly, it is desirable to provide an implant technique that provides a sufficient bone-implant interface.

### SUMMARY

Various embodiments of implant systems and related apparatus, and methods of using the same are described. In one embodiment, provided is an orthopedic implant that includes an implant body having a bone contact surface to be in contact or near contact with a bone structure during use, wherein the bone contact surface has a bone interface structure protruding therefrom. The bone interface structure includes a first elongated portion to be at least partially pressed into the bone structure during use, and a second elongated portion to be at least partially pressed into the bone structure during use. The second elongated portion is coupled to the first elongated portion and extends from the first elongated portion at an angle oblique to the first elongated portion.

In another embodiment, provided is a method that includes providing an orthopedic implant. The implant includes an implant body having a bone contact surface to be in contact or near contact with a bone structure during use, wherein the bone contact surface has a bone interface structure protruding therefrom. The bone interface structure includes a first elongated portion to be at least partially pressed into the bone structure during use, and a second elongated portion to be at least partially pressed into the bone structure during use. The second elongated portion is coupled to the first elongated portion and extends from the first elongated portion at an angle oblique to the first elongated portion. The method also includes inserting the bone interface structure into the bone structure such that that bone contact surface is in contact or near contact with the bone structure.

In another embodiment provided is and implant that includes an implant body having a bone contact surface in contact or near contact with bone structure during use and a bone interface structure protruding from the contact surface, wherein the bone interface structure includes a space truss, and wherein the bone interface structure is disposed within the bone structure during use.

In another embodiment, provided is an orthopedic implant having an implant body including a bone interface surface having a bone interface structure protruding therefrom. The bone interface structure includes a proximal portion of the bone interface structure adjacent the bone interface surface and a distal portion of the bone interface structure extending from the proximal portion of the bone interface structure, wherein the distal portion of the bone interface structure configured to be disposed at least partially into a bone structure during use.

In another embodiment, provided is an implant including an implant body and a bone interface structure having a distal bone interface structure, and a proximal bone interface structure located between the distal bone interface structure and the implant body. The distal portion of the bone interface structure to be disposed at least partially into a bone structure during use. The proximal portion of the bone interface structure is configured to be disposed in a gap between the bone structure and the implant body during use.

In another embodiment, provided is a method for providing an orthopedic implant. The method includes inserting, into a bone structure, a distal portion of a bone interface structure coupled to an implant body such that a gap is formed between the implant body and the bone structure, and wherein the gap is spanned by a proximal portion of the bone interface structure extending between the implant body and the distal portion of the bone interface structure and providing a bonding agent within the gap.

In another embodiment, provided is an orthopedic implant including an implant body including a bone interface surface having a bone interface structure protruding therefrom. The bone interface structure includes a proximal portion of the bone interface structure extending a first distance from the bone interface surface, and a distal portion of the bone interface structure extending a second distance from the bone interface surface. The second distance is greater than the first distance. The distal portion of the bone interface structure is to be disposed at least partially into a bone structure during use.

### BRIEF DESCRIPTION OF THE DRAWINGS

Advantages of the present invention will become apparent to those skilled in the art with the benefit of the following detailed description and upon reference to the accompanying drawings in which:

FIG. 1 is a block diagram that illustrates an implant in accordance with one or more embodiments of the present technique;

FIG. 2A is a diagram that illustrates a side view of the implant of FIG. 1A implanted in a bone structure in accordance with one or more embodiments of the present technique;

FIG. 2B is a diagram that illustrate a cross-sectioned view of the implant of FIGS. 1 and 2A taken across line 2B-2B in accordance with one or more embodiments of the present technique;

FIG. 3 is a diagram that illustrates a cut provided in a bone structure in accordance with one or more embodiments of the present technique;

FIG. 4 is a diagram that illustrates a cutting member in accordance with one or more embodiments of the present technique;

FIG. 5 is a diagram that illustrates a bone-implant interface including a plurality of bone interface (e.g., rod) structures provided at a contact surface of an implant in accordance with one or more embodiments of the present technique;

FIG. 6 is a diagram that illustrates an implant having bone-implant interface including a multi-layer rod-structure in accordance with one or more embodiments of the present technique;

FIGS. 7A-7G are diagrams that illustrate side views of exemplary two-dimensional rod structures in accordance with one or more embodiments of the present technique;

FIG. 8 is a diagram that illustrates an isometric view of each of the rod structures of FIGS. 7A-7G disposed on a contact surface of a bone-implant interface of an implant in accordance with one or more embodiments of the present technique;

FIGS. 9A-9B are diagrams that illustrate isometric views of a plurality of exemplary three-dimensional rod structures disposed on contact surfaces of bone-implant interfaces of implants in accordance with one or more embodiments of the present technique;

FIGS. 10A and 10B are diagrams that illustrate an isometric view and top view, respectively, of an exemplary implant in accordance with one or more embodiments of the present technique;

FIGS. 11A and 11B are diagrams that illustrate side views of knee implants in accordance with one or more embodiments of the present technique;

FIG. 12 is a diagram that illustrates a side view of an implant in accordance with one or more embodiments of the present technique;

FIG. 13 is a diagram that illustrates a shoulder implant in accordance with one or more embodiments of the present technique;

FIG. 14 is a flowchart that illustrates a method of implanting an implant in accordance with one or more embodiments of the present technique;

FIG. 15 is a block diagram that illustrates an implant in accordance with one or more embodiments of the present technique;

FIG. 16 is a block diagram that illustrates the implant of FIG. 15 implanted accordance with one or more embodiments of the present technique;

FIG. 17 is a block diagram that illustrates an implant including a truss/web interface structure in accordance with one or more embodiments of the present technique;

FIG. 18A-18C are diagrams that illustrate stops in accordance with one or more embodiments of the present technique; and

FIG. 19 is a flowchart that illustrates a method of implanting an implant in accordance with one or more embodiments of the present technique.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF ILLUSTRATIVE EMBODIMENTS

As discussed in more detail below, certain embodiments of the present technique include a system and method for implants, including orthopedic implants. In some embodiments, an implant includes a bone-implant interface that facilitates integration of the implant with adjacent bone structures. In certain embodiments, the bone-implant interface provides for effective load transfer between the implant and the adjacent bone. In some embodiments, a bone-implant interface includes a surface of the implant having an interface structure (e.g., a rod structure) extending therefrom that is to be disposed in bone structure during use. In certain embodiments, the rod structure includes a first portion extending away from the surface of the implant and a second portion oriented at least partially oblique to the first portion of the rod structure. In certain embodiments, the rod structure comprises a two dimensional structure extending from the surface. In some embodiments, the rod structure comprises one or more hook shaped members (e.g., V-shaped or U-shaped members) extending from the bone interface surface. In certain embodiments, the rod structure comprises a three dimensional structure extending from the bone interface surface. In some embodiments, the rod structure comprises a plurality of rod members coupled to one another at an apex of the orthopedic implant. In certain embodiments, the rod structure comprises two or more triangular truss structures extending from the bone interface surface, wherein two or more of the triangular truss structures (e.g., triangular planar truss units) share at least one common strut. In certain embodiments, one or more rod members of the rod structure and/or the surface of the implant include a biologic disposed thereon. In some embodiments, the rod structures are pushed into the bone during implantation. With the rods pushed into the bone the elastic nature of the bone structure may cause the bone to rebound (e.g., grow) in and around the rod structure. This may provide a “grabbing” or “holding” effect of the rod structure which enables the implants initial fixation through integration of the rod structure with adjacent bone structure. Such a grabbing or holing may inhibit movement of the implant. In certain embodiments, the implant may comprises one or more of large joint implants (e.g., a hip and/or knee implant), small joint implants (e.g., shoulder, elbow and/or ankle implants), trauma implants (e.g., shoulder fracture, long bone reconstruction implants and/or intermedullary rod implants), spine implants (e.g., fusion or dynamic implants), cranial maxi facial (e.g., jaw replacement), dental implants.

As used herein the term “truss” refers to a structure having one or more elongate struts connected at joints referred to as nodes. Trusses may include variants of a pratt truss, king post truss, queen post truss, town's lattice truss, planar truss, space truss, and/or a vierendeel truss (other trusses may also be used). Each unit (e.g., region having a perimeter defined by the elongate struts) may be referred to as a “truss unit”.

As used herein the term “planar truss” refers to a truss structure where all of the struts and nodes lie substantially within a single two-dimensional plane. A planar truss, for example, may include one or more “truss units” where each of the struts is a substantially straight member such that the entirety of the struts and the nodes of the one or more truss units lie in substantially the same plane. A truss unit where each of the struts is a substantially straight member such that the entirety of the struts and the nodes of the truss units lie in substantially the same plane is referred to as a “planar truss unit”.

As used herein the term “space truss” refers a truss having struts and nodes that are not substantially confined in a single two-dimensional plane. A space truss may include two or more planar trusses (e.g., planar truss units) wherein at least one of the two or more planar trusses lies in a plane that is not substantially parallel to a plane of at least one or more of the other two or more planar trusses. A space truss, for example, may include two planar truss units adjacent to one another (e.g., sharing a common strut) wherein each of the planar truss units lie in separate planes that are angled with respect to one another (e.g., not parallel to one another).

As used herein the term “triangular truss” refers to a structure having one or more triangular units that are formed by three straight struts connected at joints referred to as nodes. For example, a triangular truss may include three straight elongate strut members that are coupled to one another at three nodes to from a triangular shaped truss. As used herein a “planar triangular truss” is a triangular truss structure where all of the struts and nodes lie substantially within a single two-dimensional plane. Each triangular unit may be referred to as a “triangular truss unit”. A triangular truss unit where each of the struts is a substantially straight member such that the entirety of the struts and the nodes of the triangular truss units lie in substantially the same plane is referred to as a “planar triangular truss unit”. As used herein a “triangular space truss” is a space truss including one or more triangular truss units.

As used herein the term “rod” refers to an elongated member. A rod may include cross-sectional shape of varying geometries, such as a circular, oval, triangular, square, rectangular, pentagonal, or the like. A rod may include a longitudinal axis that is straight, substantially straight or curved along its length. As used herein the term “strut” refers to a rod that forms at least a portion of a truss.

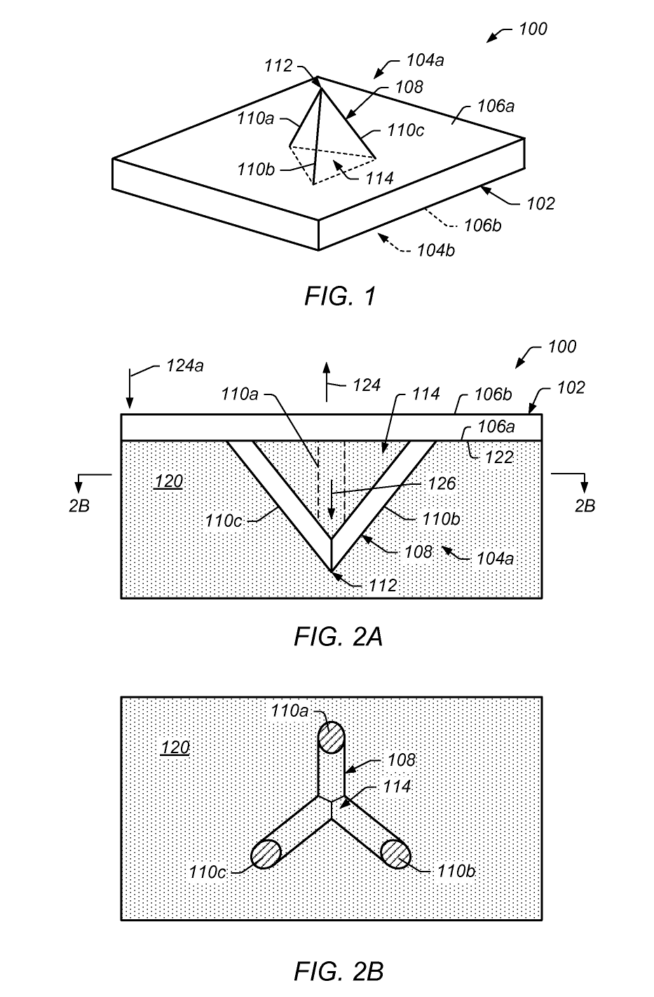

Turning now to the figures, FIG. 1 is a block diagram that illustrates an implant 100 in accordance with one or more embodiments of the present technique. In some embodiments, implant 100 may include a large joint implant (e.g., a hip and/or knee implant), a small joint implant (e.g., shoulder, elbow and/or ankle/foot implants), trauma implants (e.g., shoulder fracture, long bone reconstruction implants and/or intermedullary rod implants), a spine implant (e.g., fusion or dynamic implants), cranial maxi facial implant (e.g., jaw replacement), a dental implant, or the like. In some embodiments, implant 100 may include an intervertebral implant to be implanted between end plates of two adjacent vertebras during a spinal implant procedure. For example, implant 100 may include a fusion implant (e.g., a fusion cage) intended to rigidly fix the relative positions of two adjacent vertebrae, or and dynamic intervertebral device intended to couple to each of the two adjacent vertebrae and to facilitate motion (e.g., flexion, extension, and/or lateral bending) between the two adjacent vertebrae. In some embodiments, implant 100 may include one or more portions of an articulating knee implant. For example, implant 100 may include an upper or lower portion of a knee implant that articulate relative to one another during use, where one or both of the upper and lower portions include bone-implant interfaces that couple implant 100 to bone structures of the knee.

In some embodiments, implant 100 may include one or more bone-interfaces. For example, in the illustrated embodiment, implant 100 includes an implant body 102 having an upper bone-implant interface 104a and a lower bone-implant interface 104b. Implant 100 may include any number of bone-implant interfaces that provide for interface of the implant with bone structure. In some embodiments, upper bone-implant interface 104a may contact and secure to a first adjacent bone structure during use and lower bone-implant interface 104b may contact and secure to a second adjacent bone structure during use. For example, where implant 100 is sandwiched between two adjacent bone structures (e.g., end plates of two adjacent vertebrae), upper bone-implant interface 104a may couple to a portion of the first bone structure disposed above implant 100 and lower bone-implant interface 104b may couple to the second bone structure disposed below implant 100. It will be appreciated that the number and orientation of bone-implant interfaces for a given implant may vary based on the intended applications, and, thus, relative terms such as upper and lower are intended as exemplary and are not intended to be limiting. For example, one or both of the upper and lower bone-implant interfaces 104a and 104b may be oriented such that the are disposed laterally (e.g., as right, left, back and/or front sides of implant body 102). The box-like shape of body 102 is intended to be exemplary and is not intended to be limiting. Body 102 may include any desirable implant construct for the given implant application. For example, spinal implants or knee implants may include a shape, components, and a mechanical construct that provides for motion preservation.

In some embodiments, bone-implant interfaces 104a and 104b may include a contact surface. As used herein, the term “contact surface” refers to a portion of an implant intended to be in contact or near contact with an adjacent structure (e.g., a bone structure) and/or to adhere/couple with the adjacent structure when implanted. A contact surface may include an interface plate of an implant, for instance. In the illustrated embodiment, bone-implant interfaces 104a and 104b include an upper contact surface 106a and a lower contact surface 106b, respectively. Contact surfaces 106a and 106b may include portions of implant 100 that are intended to abut and/or integrate with adjacent bone structure when implant 100 is implanted. In some embodiments, implant 100 may include a single contact surface or more than two contact surfaces. Contact surface(s) may take any suitable shape (e.g., a substantially flat planar surface, a curved/contoured surface, ridges, or the like).

In some embodiments, bone-implant interfaces may include a structure that facilitates coupling of implant 100 to adjacent bone structure. For example, in the illustrated embodiment, upper bone interface 104a includes contact surface 106a a rod structure 108 extending therefrom. During use rod structure 108 may be pressed into adjacent bone structures. For example, implant 100 may be pressed against a bone structure such that rod structure 108 penetrates into the bone structure and contact face 106a is pressed against a corresponding surface the bone structure. Thus, rod structure 108 may be disposed in the bone structure as discussed in more detail below with respect to FIGS. 2A and 2B.

In some embodiments, some or all of the bone-implant interfaces of an implant may include one or more rod structures. For example, in the illustrated embodiment, upper bone-implant interface 104a includes a rod structure 108 disposed thereon. It will be appreciated that although rod structure 108 is illustrated on a single contact surface 106a of a single bone-implant interface 104a, other embodiments may include any number of rod structures disposed at any number of bone-implant interfaces and contact surfaces. For example, in some embodiments, implant 100 may include one or more rod structures disposed on one or both of upper and lower contact surfaces 106a and 106b of bone-implant interfaces 104a and 104b, respectively. Rod structures 108 disposed on both of upper and lower contact surfaces 106a and 106b may be of particular use where implant 100 is intended to span a gap/distance between two adjacent bone structures (e.g., implant 100 is sandwiched between the end plates of two adjacent vertebrae as discussed above).

In some embodiments, a rod structure includes one or more rod members (e.g., struts) that extend from a respective contact surface and define region (e.g., an opening or at least a partial opening) that enables bone through growth to facilitate coupling of the rod structure and, thus the implant, to the bone structure. For example, in the illustrated embodiment, rod structure 108 includes a space truss formed of three struts 110a, 110b and 110c. Struts 110a, 110b and 110c may each include substantially straight elongate rod members having a first end coupled to contact surface 106a and a second end coupled to each of the other struts at a vertex 112. Each face of the triangular shaped truss structure includes a planar truss unit having a triangular opening with a perimeter defined by two of struts 110a, 110b and 110c and the adjacent portion of contact face 106a.

As depicted, rod structure 108 includes a generally triangular shaped space truss that defines a multi-sided (e.g., three sided), substantially open region (e.g., opening/volume) 114. In some embodiments, opening/volume 114 may facilitate bone growth through rod structure 108, thereby enhancing coupling and integration of implant 100 to the adjacent bone structure. For example, at least a portion of rod structure 108 may be in contact or near contact with the adjacent bone structure, thereby enabling bone growth to extend into and/or through at least a portion of opening/volume 114 of truss structure 108 such that the bone growth interlocks with one or more struts 110a, 110b or 110c of rod structure 108. Interlocking of the bone growth and the struts 110a, 110b or 110c may rigidly fix implant 100 in a fixed location relative to the bone structure.

FIG. 2A illustrates a side view of implant 100 of FIG, 1 implanted in a bone structure 120 in accordance with one or more embodiments of the present technique. FIG. 2B illustrates a cross-sectioned view of implant 100 implanted in a bone structure 120 of FIG. 2A taken across line 2B-2B in accordance with one or more embodiments of the present technique. In the illustrated embodiment, rod structure 108 is disposed into bone structure 120 and contact surface 106a is pressed into contact with face 122 of bone structure 120. Bone structure 120 is disposed in volume 114 of rod structure 108. In some embodiments, bone structure 120 may include bone through growth that grows around struts 110a, 110b and 110c and into opening/volume 114. In some embodiments, bone structure 120 may include bone growth that encloses slits that are created in bone structure 120 during implanting of rod structure into bone structure 120. As discussed above, bone growth may provide for an interlock of rod structure 108 with bone structure 120 and may, thus, rigidly fix implant 100 in a fixed location relative to the bone structure 120. Rod structure 108 may effectively be ‘grabbed’ onto by the adjacent bone structure which enables integration of rod structure 108 with the adjacent bone structure 120. In the illustrated embodiment, a force in the direction of arrow 124 acting upon implant 100 may be counteracted by a force in the direction of arrow 126 provided by bone structure 120 resisting movement of implant 100. For example where implant 100 includes a knee implant force 124 may represent an “uplift” force. In some embodiments, a net uplift may be the result of forces acting at a particular portion of implant. For example, uplift may be the result of a downward force on implant 100 as represented by arrow 124a. In response to separating forces, such as those exerted in the direction of arrow 124, bone structure 120 coupled to rod structure 108 and provided in volume 114 may inhibit implant 100 from moving upward in the direction of arrow 124. Similar resistance to lateral/shearing forces (e.g., side to side motion, rotation motion, etc.) may be provided by bone structure 120 coupled to rod structure 108 and provided in opening/volume 114. The load transfer to bone structure 120 in volume 114 through the pulling of strut 110b and 110c in the direction of force 124 may encourage an increase in bone density through remodeling principles found in previously mentioned Wolfs law. In some embodiments, coupling of surface to bone structure 120 (e.g., enhanced via use of a biologic or porous coating) may also provided resistance to motion of implant 100 relative to bone structure 120.

In some embodiments, rod structure 108 may extend from contact surface 106a by a distance (e.g., height) that is less than, about the same, the same, or greater than a height/thickness of a body 102 of implant 100. For example, in the illustrated embodiment, rod structure 108 protrudes extends a distance that is about four times the height/thickness of implant body 102. In some embodiments, rod structure 108 may have a height that is about 10%, 15%, 20%, 30%, 40%, 50%, 60%, 70%, 80%, 90%, 100%, 150%, 200%, 250%, 300%, 350%, 400%, 450%, 500%, 550% that of body 102 of implant 100. In some embodiments, rod structure 108 may have a height that is about 1 mm, 2 mm, 3 mm, 4 mm, 5 mm, 10 mm, 15 mm, 20 mm, 25 mm, 30 mm, 40 mm, 45 mm, 50 mm, 55 mm, 60 mm, 65 mm, 70 mm, 75 mm, 80 mm or greater.

In some embodiments, implant 100 may be pressed into contact with the adjacent bone structure such that at least a portion of rod structure 108 is disposed inside of the adjacent bone structure upon implantation. For example, in some embodiments, implant 100 may be pressed into contact with bone structure 120 such that vertex 112 pierces into the bone structure and is advanced such that at least a portion of struts 110a, 110b or 110c and opening/volume 114 extend into bone structure 120. Such a technique may encourage bone to grow into and/or through opening/volume 114. In some embodiments, implant 100 may be advanced/pressed into bone structure 120 until the respective contact surface (e.g., upper contact surface 106a) is in contact or near contact with surface 122 of bone structure 120.

In some embodiments, at least a portion of a bone-implant interface (e.g., the rod structure and/or the contact surfaces) may be coated/treated with a material intend to promote bone growth and/or bone adherence and/or an antimicrobial to prevent infection via the rod structure and/or the contact surface. In some embodiments, at least a portion of a bone-implant interface may be coated with a pain medication (e.g., analgesics) to reduce pain after insertion of the implant into the bone. For example, in some embodiments, at least some or all of the surfaces of struts 110a, 110b or 110c and/or contact surfaces 106a and 106b may be coated with a biologic, a bone growth factor and/or pain medication. In some embodiments, some or all the bone-implant interface (e.g., the rod structure and/or the contact surfaces) may include a porous surface/coating that facilitates adherence of the contact surface to the adjacent bone structure. For example, some or all of struts 110a, 110b and 110c and/or contact surfaces 106a and 106b may include a porous surface texture to promote bone growth that adheres to rod structure 108 and contact surfaces 106a and 106b.

In some embodiments, at least a portion of the adjacent bone structure in which a rod structure is to be implanted may be pierced/cut/slit prior to the rod structure being advanced/pressed into the adjacent bone structure. For example, a bone end plate of a vertebra may be cut to accept struts 110a, 110b and 110c of rod structure 108. In some embodiments, a cutting tool/edge may be used to cut into the adjacent bone structure such that the resulting cuts accommodate portions (e.g., one or more struts or rods) of rod structure 108. For example, where rod structure 108 includes a triangular shape, such as that depicted in FIGS. 1-2A, one or more complementary cuts may be made into the adjacent bone structure in a complementary Y-shaped pattern.

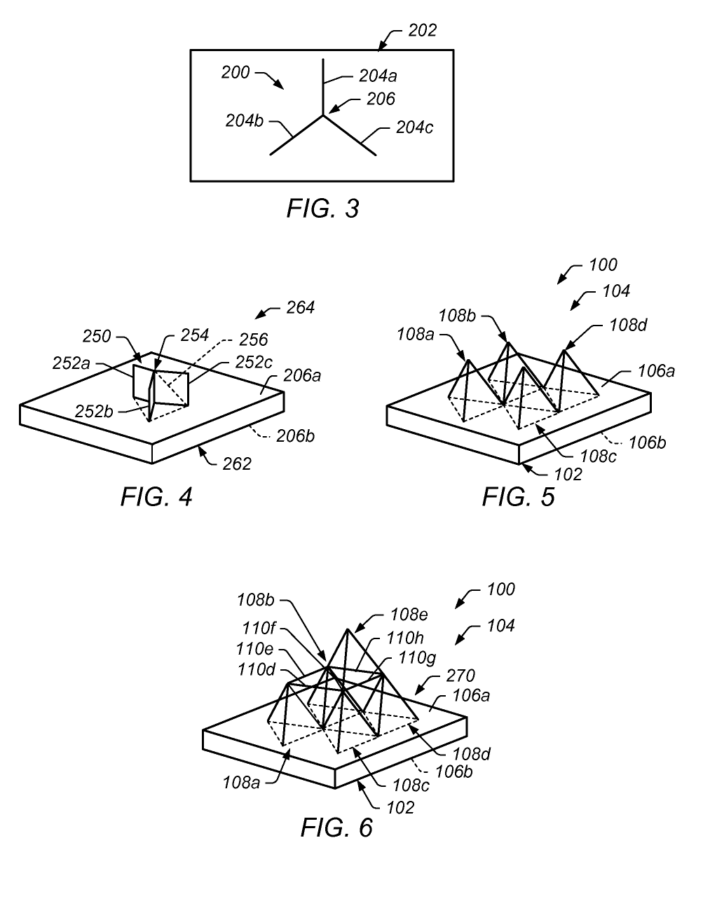

FIG. 3 illustrates a cut 200 that may be provided in a bone structure 202 in accordance with one or more embodiments of the present technique. Bone structure 202 may be similar to bone structure 120. Cut 220 may be provided prior to or as a result of rod structure 108 being advanced/pressed into the adjacent bone structure 202. FIG. 3 may be representative of an end view of a bone structure. For example, FIG. 3 may be illustrative of the face of a vertebra end plate and a Y-shaped cut extending into the face (e.g., looking upward/downward into the end plate of the vertebrae) that is shaped to accept at least a portion of rod structure 108. In some embodiments, cut 200 may include one or more segments intended to accommodate one or more portions (e.g., struts or rods) of a rod structure. For example, in the illustrated embodiment, cut 200 includes three slits 204a, 204b and 204c formed in bone structure 202. Slits 204a, 204b and 204c may extend from the face of bone structure 202 into bone structure 202 in a direction substantially perpendicular to a face of bone structure 202 and/or substantially parallel to the intended direction of advancement of struts of rod structure 108 and/or implant 100 into bone structure 202.

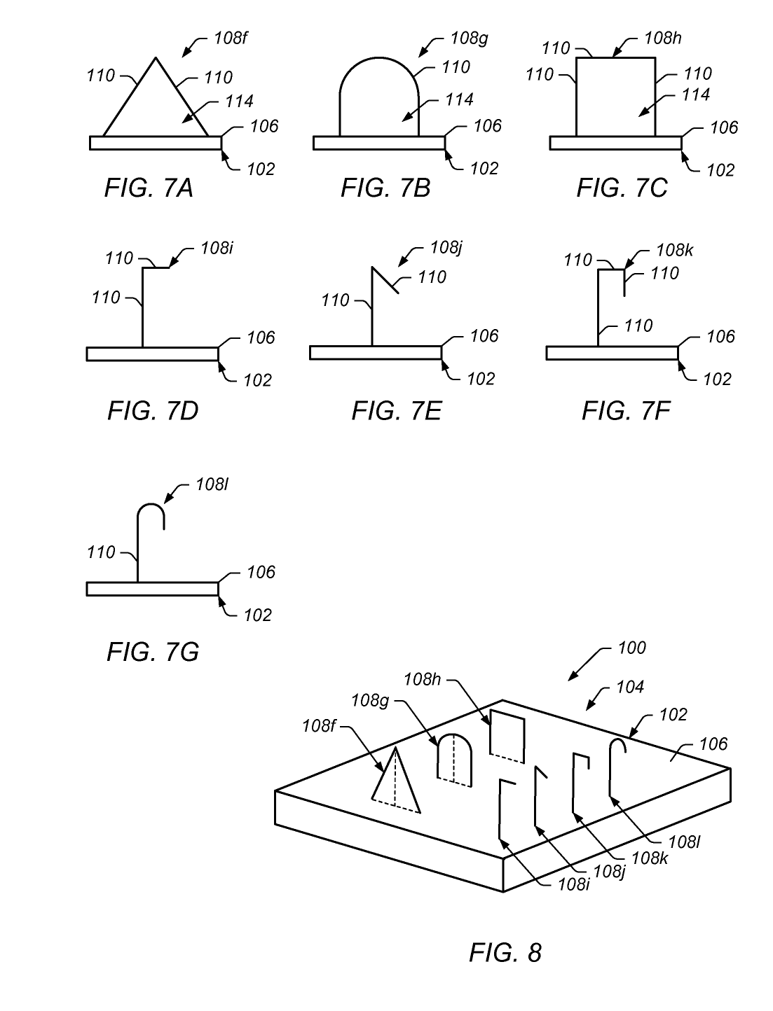

In some embodiments, slits include cuts into the bone that do not require any bone material to be removed. For example, a sharp cutting edge (e.g., a knife/blade) may be advanced into bone structure 202 to create slits 204a, 204b and 204c, with out removing any bone structure 202 or a substantial amount of bone structure 202. During implantation of implant 100 into bone structure 202, struts 110a, 110b or 110c may slide into slits 204a, 204b and 204c, respectively. Cut 200 may be complementary to the shape/orientation of portions (e.g., rods or struts) of rod structure 108. Although the illustrated embodiments includes three slits oriented at approximately one-hundred twenty degrees relative to one another about a vertex 206, other embodiments may include any number of slits in any variety of orientation to accommodate one or more struts of a rod structure extending from a contact face of an implant. For example, where rod structure 108 is substantially pyramidal in shape (e.g., see rod structure 108g described below with respect to FIG. 7), cut 200 may include four slits oriented at approximately ninety-degrees relative to one another.

In some embodiments, cut 200 may be formed by one or more complementary cutting members (e.g., knives/blades) that are pressed, slid, or otherwise advanced into bone structure 202. In some embodiments, a cutting member includes one or more cutting edges arranged complementary to the profile of the portions (e.g., rods or struts) of rod structure 108 such that advancement of the cutting edge cuts one, a plurality, or all of the slits to accommodate rod structure 108 being advanced/pressed into the bone structure.

FIG. 4 illustrates a cutting member 250 in accordance with one or more embodiments of the present technique. Cutting member 250 may include three cutting blades 252a, 252b and 252c oriented at approximately one-hundred twenty degrees relative to one another about a vertex 254. In some embodiments, cutting members 252a, 252b and 252c, are arranged complementary to slits 204a, 204b and 204c of cut 200 and/or struts 110a, 110b or 110c of rod structure 108. Although the illustrated embodiment includes three cutting blades oriented at approximately one-hundred twenty degrees relative to one another about a vertex 254, other embodiments may include any number of cutting blades in any variety of orientations to accommodate one or more portions (e.g., rods or struts) of rod structure 108 of implant 100. For example, where rod structure 108 is substantially pyramidal in shape (e.g., see rod structure 108b described below with respect to FIG. 7), cutting member 250 may include four cutting blades oriented at approximately ninety-degrees relative to one another.

In some embodiments, the cutting blades of cutting member 250 may be advanced into bone structure 202 at a depth that is about the same or deeper than a height of rod structure 108. In some embodiments, the cutting blades may be advanced into bone structure 202 at a depth that is about the same or shallower than a height of rod structure 108. In some embodiments, a leading edge of the cutting blades may be shaped to be complementary to the shape of the struts. For example, the leading edge of one, a plurality, or all of cutting blades 252a, 252b and 252c, may be angled similar to the angle of struts 110a, 110b or 110c extending from contact surface 106a, as illustrated by dashed line 256 which includes an angle substantially similar to that of a corresponding strut 110c of implant 100.

In some embodiments, cutting member 250 may be provided as an instrument that is advanced into bone structure 202. In some embodiments, cutting member 250 may be integrated with or more other devices used during the implantation procedure. For example, during a spinal implant procedure, cutting member 250 may be coupled to a distractor typically positioned between the vertebrae and expanded to set the relative positions of adjacent vertebrae. The force of distraction may act to advance cutting member 250 into bone structure 202. FIG. 4 illustrates cutting member 250 disposed on a top surface 260a of a body 262 of a distractor 264, in accordance with one or more embodiments of the present technique. In some embodiments, one or more cutting members may be disposed on other portions of an instruments (e.g., distractor 264), such as a bottom surface 206b. Where distractor 264 includes a distractor (e.g., a spinal distractor), during use, distractor 264 may be disposed between the adjacent bone structures (e.g., adjacent vertebrae) and expanded such that top and bottom surfaces 260a and 260b move away from one another, thereby pressing one or more of cutting members 250 (e.g., on top and/or bottom contact surfaces 260a and 260b) into the adjacent bone structure (e.g., 202) to form one or more cuts (e.g., cut 200) into the bone structure (e.g., end plates of the adjacent vertebrae), where the cuts are intended to accommodate struts (e.g., struts 110a, 110b and 110c) of the rod one or more structures (e.g., rod structure 108) of an implant (e.g., implant 100) to be engaged with the bone structure (e.g., bone structure 120 or 202). In some embodiments, a distractor may be used to increase a separation distance between two adjacent bone structures (e.g., between end plates of adjacent vertebrae). In some embodiments, subsequent to making cuts, the distractor is unexpanded and/or removed, and the implant (e.g., 100) is disposed between the bone structures (e.g., in substantially the same position as the distractor) such that one or more rod structures are aligned/engaged with one or more of the resulting cuts. Other embodiment may include pressing or otherwise advancing cutting member 250 into a bone structure where a rod structure is to be disposed.

Although several of the above embodiments have been described with regard to a single rod structure, other embodiments may include any number and configurations of rod structures. In some embodiments, a plurality of rod structures may be provided at one or more bone-implant interfaces of implant 100. FIG. 5 depicts bone-implant interface 104 including a plurality of rod structures 108a, 108b, 108c and 108d provided at upper contact surface 106a of implant 100 in accordance with one or more embodiments of the present technique. In the illustrated embodiment, four rod structures 108a, 108b, 108c and 108d are disposed substantially adjacent one another on upper contact surface 106a of implant 100. Some or all struts of rod structures 108a, 108b, 108c and 108d may share at least one common vertex with another of rod structures 108a, 108b, 108c and 108d at the contact surface 106a. In some embodiments, one, a plurality or all of rod structures may be spaced apart from one another. For example, one, a plurality, or all of rod structures 108a, 108b, 108c and 108d may not share a vertex at or near contact surface 106a. In some embodiments, any number of rod structures may be provided on any portion of implant 100. In some embodiments, the shape and orientation of the rod structures may be varied to mimic various desired shapes. For example, in some embodiments, the truss structures of rod structures 108a-108d may be varied in height and/or orientation to provide a curved profile similar to that of a ball and/or a socket of a joint.

In some embodiments, a bone-implant interface may include a plurality of rod structures stacked upon one another to form a web-like truss structure disposed on one or more contact surfaces of implant 100. FIG. 6 illustrates implant 100 having bone-implant interface 104 including a multi-layer rod-structure (e.g., truss/web structure) 270 in accordance with one or more embodiments of the present technique. Multilayer rod structure 270 may be disposed at a bone-implant interface of implant 100. For example, in the illustrated embodiment, multi-layer rod-structure 270 is disposed on contact surface 106a of implant 100. In some embodiment, a multi-layer rod-structure may include a plurality of rod structures interconnected and/or stacked upon one another. Stacking of rod structures may address complications in revision procedures where significant bone loss has occurred and there is a need to replace the bone. The first layer of the stacked design may replace the ‘height’ of the primary bone structure and can be filled with a bone void filler such as cement (e.g., para-Methoxymethamphetamine (PMMA)), hydroxiappatite, calcium phosphate which will remodel into bone over time, or the like. The second layer of the stacked structure may provide for fixation and load transferring. For example, in the illustrated embodiment, a triangular rod structure 108e is stacked atop vertices of rod structures 108b, 108c and 108d. In some embodiments, the shape and orientation of the web structure 270 may be varied to mimic various desired shapes. For example, in some embodiments, web structure 270 may be varied in height and/or orientation to provide a curved profile similar to that of a ball and/or a socket of a joint.

In some embodiments, one or more additional rod members may be provided between one, a plurality, or all of the vertices of rod structures. For example, in the illustrated embodiment, struts 110d-110h extend between vertices of rod structures 108a-108d. In some embodiments, one or more struts may extend between some or all of the struts at or near the point where they are coupled to the contact face. For example, one or more rod members/struts may extend in place of one or more of the dashed lines illustrated in FIGS. 1, 4 and 5.

Some of the above embodiments have been described with respect to a particular shaped rod structure (e.g., a triangular shaped space truss structure 108) although various shapes of truss structures are contemplated. It will be appreciated that such description is intended to be exemplary and is not intended to be limiting. For example, in some embodiments, rod structure 108 may include a web/truss structure, such as those described in U.S. Provisional Patent Application No. 61/138707 entitled “TRUSS IMPLANT” by Jessee Hunt, filed Dec. 18, 2008 and U.S. patent application Ser. No. 12/640,825 entitled “TRUSS IMPLANT” by Jessee Hunt, filed Dec. 17, 2009, which are hereby incorporated by reference as if fully set forth herein.

In some embodiments, a rod structure may include a two-dimensional rod structure. FIGS. 7A-7G illustrate side views of exemplary two-dimensional rod structures 108f-108l in accordance with one or more embodiments of the present technique. FIG. 8 illustrates an isometric view of each of rod structures 108f-108l of FIGS. 7A-7G disposed on contact surface 106 of bone-implant interface 104 of implant 100 in accordance with one or more embodiments of the present technique. FIG. 7A includes a triangular shaped rod structure 108f that includes two rod members 110 each having ends coupled to one another at a vertex and coupled to contact surface 106 of body 102, defining an opening 114 through which bone growth may occur. Rod structure 108f may include a triangular-shaped planar truss. FIG. 7B includes a U-shaped rod structure 108g that includes a curved rod member 110 having a U-shaped bend at its apex and having ends coupled to contact surface 106 of body 102, defining an opening 114 through which bone growth may occur. In some embodiments, curved rod member 110 may include two or more portions (e.g., rod members) that form the U-shape. For example, a right curved portion may extend from contact surface 106, a left curved portion may extend from contact surface 106 and the two portions may be coupled to one another at an apex of rod structure 108g. Thus, the two curved portions may be oriented relative to one another to form the U-shape defining opening 114. FIG. 7C includes a square/U-shaped structure 108h that includes a plurality of substantially straight rod members 110 having a substantially straight rod member its apex and having two substantially straight rod members at either end coupled to contact surface 106 of body 102, defining an open region 114 through which bone growth may occur. FIG. 7D includes a L-shaped rod structure 108i that includes a first a substantially straight rod member 110 extending from contact surface 106 of body 102 and a second substantially straight rod member 110 oriented at an oblique angle (e.g., substantially perpendicular angle) to the first rod member 100. FIG. 7E includes a hook/barb-shaped rod structure 108j that includes a first a substantially straight rod member 110 extending from contact surface 106 of body 102 and a second substantially straight rod member 110 oriented at an oblique angle (e.g., an acute angle of about forty-five degrees) relative to the first rod member 110. Other embodiments may include various angles of the second rod member relative to the first rod member from about ten degrees to about one hundred seventy degrees (e.g., a second rod member angled oblique about ten, twenty, thirty, forty, fifty, sixty, seventy, eighty, ninety, one hundred, one hundred ten, one hundred twenty, one hundred thirty, one hundred forty, one hundred fifty, one hundred sixty, and/or one seventy degrees relative to the first rod member). FIG. 7F includes a hook-shaped rod structure 108k that includes a first a substantially straight rod member 110 extending from contact surface 106 of body 102, a second substantially straight rod member 110 oriented at an oblique angle (e.g., substantially perpendicular angle) to the first rod member 100, and a third substantially straight rod member 110 oriented substantially parallel to the first rod member 110. Other embodiments may include various angles of the second rod member relative to the first rod member (e.g., a second member angled oblique from about ten degrees to about one-hundred seventy degrees relative to the first rod member-a second member angled oblique about ten, twenty, thirty, forty, fifty, sixty, seventy, eighty, ninety, one hundred, one hundred ten, one hundred twenty, one hundred thirty, one hundred forty, one hundred fifty, one hundred sixty, and/or one seventy degrees relative to the first member) and various angles of the third rod member relative to the second rod member (e.g., a third rod member angled oblique from about ten degrees to about one-hundred seventy degrees relative to the second rod member-a third member angled oblique about ten, twenty, thirty, forty, fifty, sixty, seventy, eighty, ninety, one hundred, one hundred ten, one hundred twenty, one hundred thirty, one hundred forty, one hundred fifty, one hundred sixty, and/or one seventy degrees relative to the second rod member). FIG. 7G includes a hook-shaped rod structure 108l that includes a rod member having a rounded end curved back towards contact surface 106 of body 102. Accordingly, rod structure 108l may include rod member 100 having a longitudinal axis that is curved at least at one end to provide a rounded bend that forms a hook-like shape. The bend may include a bend from about ten degrees to about one hundred eighty degrees, as depicted, or more.

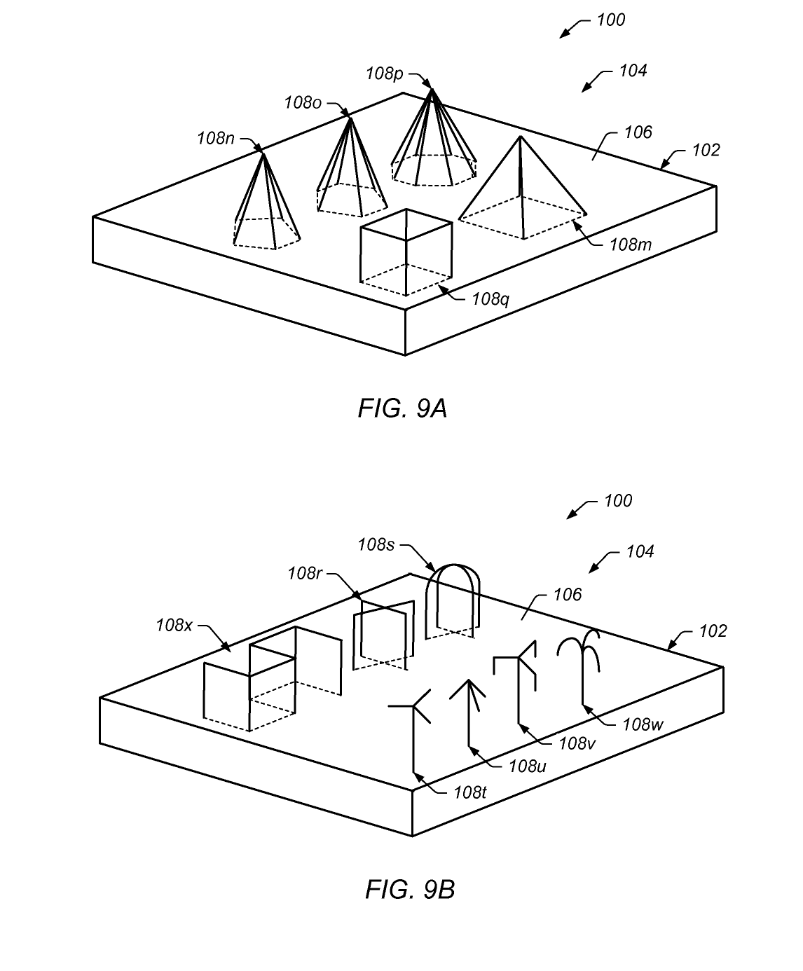

In some embodiments, a rod structure may include a three-dimensional rod structure. For example rod structure may include one or more three-sided triangular shaped space truss structure similar that of rod structure 108 described above with respect to FIGS. 1-6. FIGS. 9A-9B illustrate isometric views of a plurality of exemplary three-dimensional rod structures 108m-108w disposed on contact surfaces 106 of bone-implant interfaces 104 of implants 100 in accordance with one or more embodiments of the present technique. Rod structures 108m, 108n, 108n and 108p include a four-sided (e.g., pyramidal) space truss, five-sided space truss, six-sided space truss, and an eight sided space truss, respectively. Rod structure 108q includes a rectangular/square shaped rod structure formed from a plurality of rod members similar to those of rod structure 108h of FIG. 7C. Rod structure 108r includes an X-shaped rod structure formed from a plurality of rod members similar to those of rod structure 108h of FIG. 7C. Rod structure 108s includes an X-shaped rod structure formed from a plurality of curved rod members similar to those of rod structure 108g of FIG. 7B. Rod structure 108t includes a three hook (e.g., treble-hook) shaped rod structure formed from a plurality of rod structures similar to those of rod structure 108i of FIG. 7D. Rod structure 108u includes a treble-hook shaped rod structure formed from a plurality of rod structures similar to those of rod structure 108j of FIG. 7E. Rod structure 108v includes a treble-hook shaped rod structure formed from a plurality of rod structures similar to those of rod structure 108k of FIG. 7F. Rod structure 108w includes a treble-hook shaped rod structure formed from a plurality of rod structures similar to those of rod structure 108l of FIG. 7G. Rod structure 108x includes an S-shaped rod structure formed from a plurality of rod members similar to those of rod structure 108h of FIG. 7C disposed in a repetitive pattern. Other embodiments may include a random pattern and/or may include a pattern that includes some or all of the other shapes and arrangements of rod structures (e.g., 108-108w) described herein.

In some embodiments, any of the rod structures described herein may be formed via coupling of a plurality of rod members or may be formed of a single rod member that is bent/formed/molded into the provided shape. In some embodiments, rod members (e.g., struts) may have thickness (e.g., diameter) between about 0.25 millimeters (mm) and 5 mm (e.g., a diameter of about 0.25 mm, 0.5 mm, 0.6 mm, 0.7 mm, 0.8 mm, 0.9 mm, 1 mm, 2 mm, 3 mm, 4 mm, or 5 mm). In some embodiments, a rod structure may have an overall length or width of less than about 1 inch (e.g., a length less than about 0.9 in, 0.8 in, 0.7 in, 0.6 in, 0.5 in, 0.4 in, 0.3 in, 0.2 in, 0.1 in).

Embodiments may include rod structures having any variety of shapes. For example, other embodiments may include a seven sided space truss and/or space trusses having more than eight sides. In some embodiments, any type, size, number, or combination of number, types and sizes of rod structures may be provided on one, a plurality, or all of the contact faces of an implant.

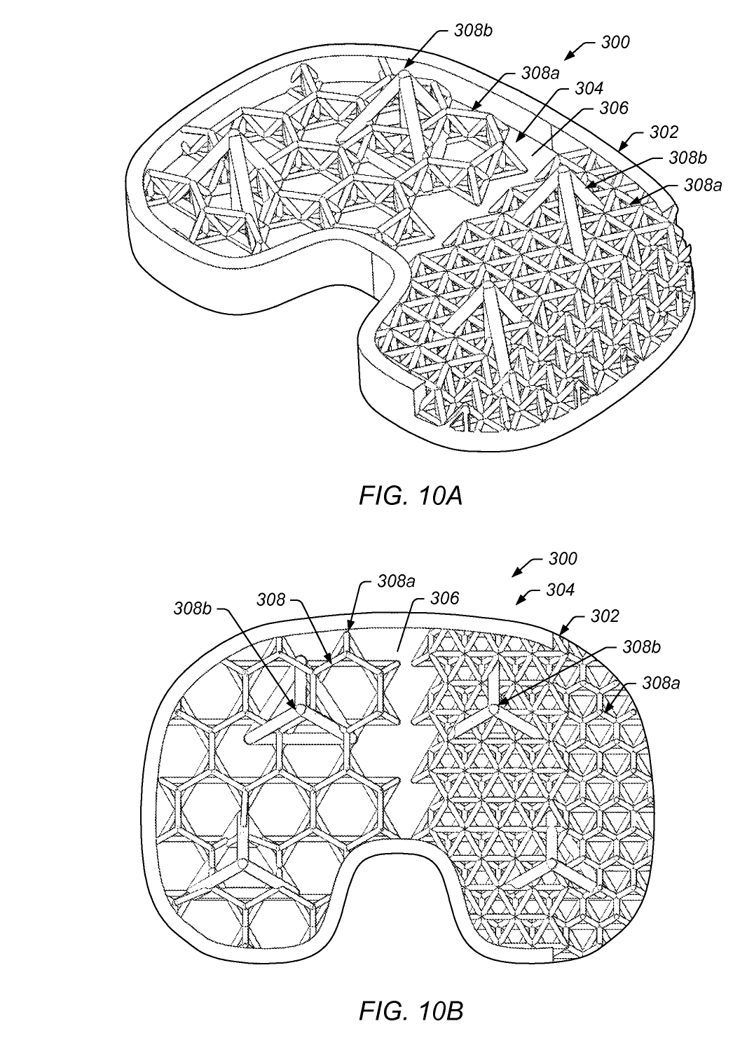

FIGS. 10A and 10B illustrate an isometric view and top view, respectively, of an exemplary implant 300 in accordance with one or more embodiments of the present technique. In some embodiments, implant 300 includes a body 302 having a bone-implant interface 304 that includes a contact face 306 having a plurality of rod structures 308 extending therefrom. In the illustrated embodiment, rod structures 308 include a plurality of different shapes and sizes. For example some of rod structures 308 include smaller sized triangular shaped space trusses, some of rod structures 308 include larger sized triangular shaped space trusses that extend between and above rod members of the smaller sized space trusses, and some of the rod structures 308 include struts arranges in a hexagonal pattern to form six-sided planar trusses of corresponding space trusses. In some embodiments, implant 300 may include a lower portion of a knee implant.

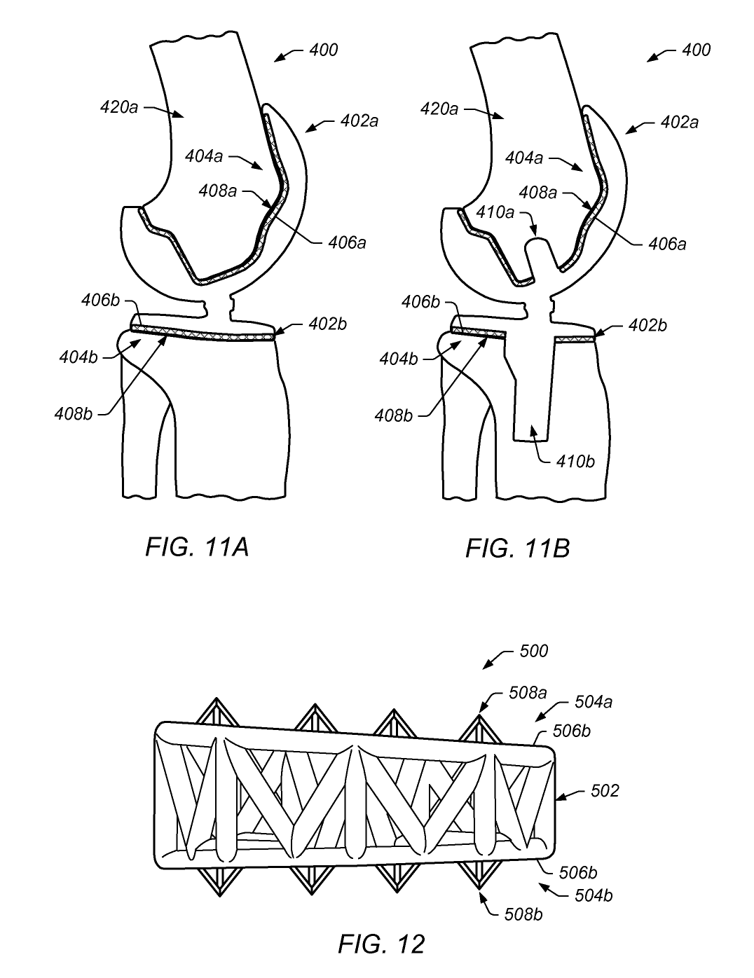

FIG. 11A illustrates a side view of a knee implant 400 in accordance with one or more embodiments of the present technique. In the illustrated embodiment, implant 400 includes an upper body 402a and a lower body 402b having bone-implant interfaces 404a and 404b respectively. Upper body 402a includes a cup that cradles bone structure 420a. In some embodiments, one or both of bone-implant interfaces 404a and 404b may include a rod structure. For example, in the illustrated embodiment, interfaces 404a and 404b include rod structures 408a and 408b extending from contact surfaces 406a and 406b, respectively.

In some embodiments, rod structures may be used in conjunction with other forms and types of bone-implant interfaces, such as a rod or keel. For example, as depicted in FIG. 11B, upper body 402a may include an elongated rod 410a that is disposed into bone structure 420a and/or lower body 410 may include an elongated rod 410b that is disposed into bone structure 420b. Elongated rod may include a dowel rod, screw, keel or the like.

In some embodiments, implant 100 (e.g., implant body 102) may include a web/truss structure, such as those described in U.S. Provisional Patent Application No. 61/138,707 entitled “TRUSS IMPLANT” by Jessee Hunt, filed Dec. 18, 2008 and U.S. patent application Ser. No. 12/640,825 entitled “TRUSS IMPLANT” by Jessee Hunt, filed Dec. 17, 2009, which are hereby incorporated by reference as if fully set forth herein FIG. 12 illustrates a side view of an implant 500 in accordance with one or more embodiments of the present technique. In the illustrated embodiment, implant 500 includes a body 502 having web/truss structure, and upper and lower bone-implant interfaces 504a and 504b that include a plurality of rod structures 508a and 508b extending from upper and lower contact surfaces 506a and 506b, respectively, of implant 500. Implant 500 may include a spinal implant (e.g., spinal fusion cage, vertebral body replacement (VBR) or spinal motion preservation implant) in some embodiments. For example, upper bone-implant interface 504a may integrate with an endplate of an upper vertebrae (e.g., rod structures 508a may be pressed in the endplate of the upper vertebrae) and lower bone-implant interface 504b may integrate with an endplate of a lower vertebrae (e.g., rod structures 508b may be pressed in the endplate of the vertebrae).

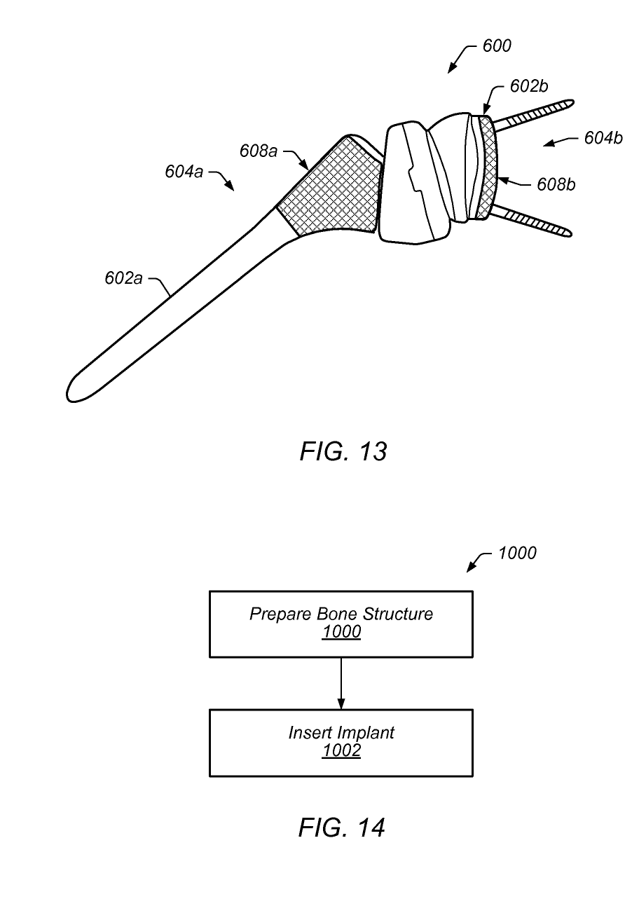

FIG. 13 is a diagram that illustrates a shoulder implant 600 in accordance with one or more embodiments of the present technique. In the illustrated embodiment, implant 600 includes a first body 602a and a second body 602b having bone-implant interfaces 604a and 604b respectively. In some embodiments, one or both of bone-implant interfaces 604a and 604b may include a rod structure. For example, in the illustrated embodiment, interfaces 604a and 604b include rod structures 608a and 608b. In some embodiments, only a rod interface is used to interface with bone. For example, the elongated portion of body 602a may not be present and/or the screws of body 602b may not be present.

FIG. 14 is a flowchart that illustrates a method 1000 of implanting an implant in accordance with one or more embodiments of the present technique. In the illustrated embodiment, method 1000 includes preparing a bone structure, as depicted at block 1002, and inserting an implant (e.g., implant 100), as depicted at block 1002. In some embodiments, preparing a bone structure includes positioning the bone structure. For example, a distractor (e.g., distractor 262 of FIG. 4) may be used to separate adjacent bone structures such that the implant can be sandwiched between the two adjacent bone structures. In some embodiments, preparing a bone structure includes cutting/slitting the bone structure to accommodate one or more struts of a rod structure of an implant to be coupled to the bone structure. For example, a cutting member (e.g., cutting member 250) may be advanced into the bone structure to create a cut (e.g., cut 200) including one or more slits (e.g., slits 204a, 204b and 204c). In some embodiments, distraction and cutting may be provided simultaneously via use of a distractor that includes one or more cutting members coupled to one or more of its contact faces (e.g., distractor 264 having cutting members 250 coupled to both upper and lower faces 206a and 206b).

In some embodiments, inserting the implant includes positioning the implant (e.g., implant 100) adjacent the bone structure (e.g., bone structure 202), aligning the rod structure (e.g., rod structure 108) with a complementary portion of the bone structure (e.g., cut 200) and/or advancing bone-implant interface (e.g., bone-implant interface 104, 104a or 104b) toward the bone structure such that at least the rod structure is in contact or near contact with the bone structure. In some embodiments, the implant may be advanced until the contact surface (e.g., contact surfaces 106a and/or 106b) is in contact or near contact with the bone structure, such that at least portion or substantially all of the rod structure is disposed in the bone structure. For example, substantially all of the struts of the truss structure 108 may be disposed in the slits 204a, 204b and 204c provided in the bone structure.

As will be appreciated, method 1000 is exemplary and is not intended to be limiting. One or more of the elements described may be performed concurrently, in a different order than shown, or may be omitted entirely. Method 1000 may include any number of variations. For example, in some embodiments, rod/struts of rod structure 108 may include a sharp/thin profile such that minimal preparation of the bone structure needed (e.g., cuts do not need to be provided in the bone structure) as the struts of the rod structure may pierce/slice the bone structure as the implant is advanced into contact with the bone surface. Accordingly, in some embodiments, steps 1002 and 1004 of method 1000 may be combined into a single step.

In some embodiments, an implant may be positioned such that a gap is provided between a body (e.g., interface surface) of the implant and a surface of the boney structure. The gap, for example, may include a region in which a filler (e.g., a bone void filler, cement, or bone graft material) is provided to adhere/secure the implant body to the boney surface. In some embodiments, an implant includes a body having a bone interface structure extending therefrom, and may be positioned such that at least a distal portion of a bone interface structure is inserted into bone structure resulting in a gap between the body and the boney structure. The gap may be spanned by a proximal portion of the bone interface structure that extends between the body and the distal portion of the bone interface structure. During use, the gap may be impregnated or otherwise filled with a filler (e.g., a bone void filler, cement, or bone graft material) that provides for adhesion/bonding of the implant body to the boney structure. For example, filler may be injected into the web interface structure prior to, during, or after insertion of the distal portion of the bone interface structure into the boney structure.

The proximal portion of the bone interface structure may include one or more struts (e.g., a web structure) that may act as reinforcing members that help to increase the strength and durability of the bond between the implant body and the boney structure. The combination of the bone interface structure extending into the boney surface and the gap spanned by bone interface structure and impregnated with filler (e.g., a bone void filler, cement, or bone graft material) may provide a strong and durable coupling of the implant to the boney structure and/or may fill voids of boney structure at the implant location. For example, in the case of a knee implant, weight-bearing surfaces of the knee joint that are removed may be accounted for by the proximal portion of the bone interface structure that spans the gap between the implant body and the surface of the bone, as well as the height of the implant body.

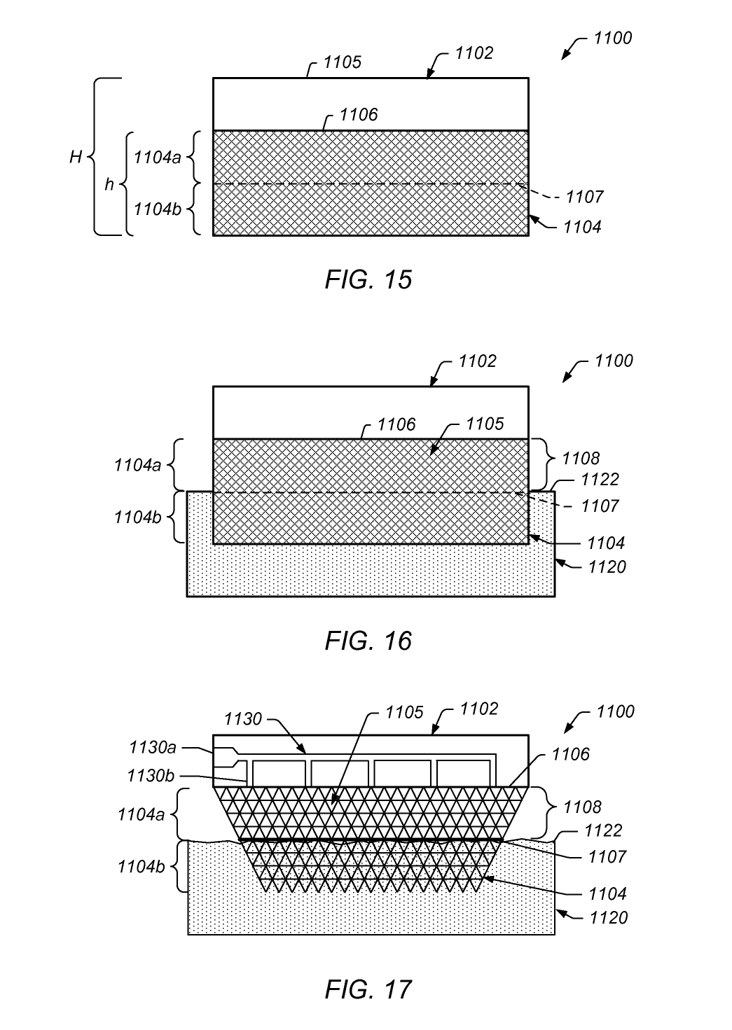

FIG. 15 is a block diagram that illustrates an implant 1100 in accordance with one or more embodiments of the present technique. Implant 1100 may include any of the implants described herein. Implant 1100 may include a cemented implant. For example, implant 1100 may include a knee implant that is at least partially coupled to the boney structure of the knee via cement. Implant 1100 includes an implant body 1102 having a bone interface structure 1104 extending from an interface surface 1106 of implant 1100. Implant body 1102 may be similar to that of implant body 102 described herein. For example, implant body 1102 may include a body of a spinal implant, a knee implant, an ankle/foot implant, a shoulder implant, or the like. The bone interface structure 1104 may have a height that is about 5%, 10%, 15%, 20%, 30%, 40%, 50%, 60%, 70%, 80%, 90%, 100%, 110%, 120%, 130%, 140%, 150%, 160%, 170%, 180%, 190% 200% or more of a height of the implant body 1102.

Surface 1106 may be similar to that of surface 106 described herein. Surface 1106 may include an external surface of a body of implant 1100. Surface 1106 may take any suitable shape (e.g., a substantially flat planar surface, a curved/contoured surface, ridges, or the like). During use, surface 1106 may remain spaced apart from an adjacent bone structure such that a gap exits between the two. For example, some or all of surface 1106 may not be in contact or near contact with the adjacent boney structure, thereby forming gap there between. Although bone interface structure 1104 is depicted as extending from a single/lower surface of implant 1100, other embodiments may include similar bone interfaces 104 extending from a plurality or all of the surfaces of implant 1100. For example, bone interface structures may extend from both of upper surface 1105 and lower/interface surface 1106 of implant 1100.

In the illustrated embodiment, bone interface structure 1104 extends from surface 1106. Structure 1104 includes a proximal portion 1104a and a distal portion 1104b. Proximal portion 1104a may include a first portion/layer of the structure coupled to surface 1106. Distal portion 1104b may include a second portion/layer of structure 1104 that is inserted into bone structure during use. Structure 1104 may include a contiguous structure formed or one or more struts, as described herein. Structure 1104 may include a biologic, growth factor or pain medication disposed thereon. When installed into bone, structure 1104 may inhibit uplift, migration, rotation or other movement of implant 1100 relative to the bone structure.

For example, structure 1104 may include a web/truss structure, such as those described in U.S. Provisional Patent Application No. 61/138,707 entitled “TRUSS IMPLANT” by Jessee Hunt, filed Dec. 18, 2008 and U.S. patent application Ser. No. 12/640,825 entitled “TRUSS IMPLANT” by Jessee Hunt, filed Dec. 17, 2009, which are hereby incorporated by reference as if fully set forth herein. Structure 1104 may include a bone-implant interface such as bone-implant interface 104, 104a, 104b 304, 404a, 404b, 504a, 504b, 604a and/or 604b described herein. For example, structure 1104a may include a web structure and/or rod structure (e.g., V-shaped structure, a U-shaped structure, and a hook-shaped structure) and structure 1104b may include a web structure and/or rod structure.

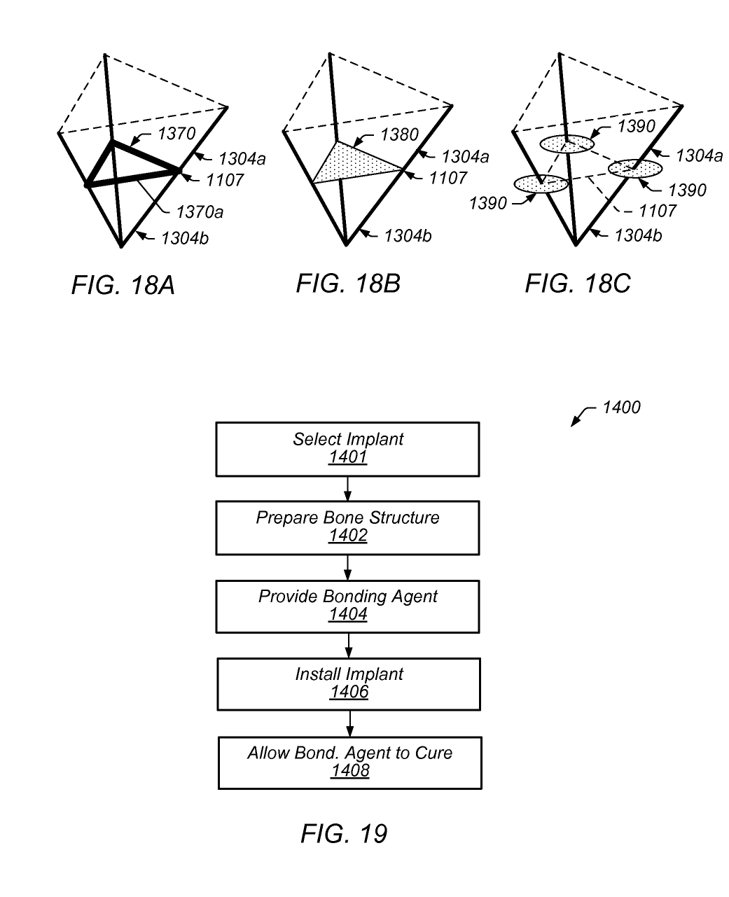

In some embodiments, proximal and distal portions 1104a and 1104b may be substantially similar. For example, proximal and distal portions 1104a and 1104b may be formed of a substantially homogeneous web structure having similar strut spacing and arrangement. In some embodiments, proximal and distal portions 1104a and 1104b may vary in one or more respects. Distal portion 1104b may include a structure that is conducive to insertion into the adjacent bone structure, and proximal portion 1104a may include a structure that at least partially inhibits insertion into the adjacent bone structure. For example, distal portion 1104b may include a relatively open web-structure having increased spacing between struts, and/or may include a reduced number and/or size of laterally oriented struts to facilitate insertion of distal portion 1104b into bone structure. Proximal portion 1104a may include a relatively closed web-structure having decreased spacing between struts (smaller than that of distal portion 1104b), and/or may include an increased number and/or size of laterally oriented struts or stops (e.g., as described in more detail below with respect to FIGS. 18A-18C) to inhibit insertion of proximal portion 1104a into the bone structure.

In some embodiments, the proximal and distal portions may be substantially independent with respect to the structural support provided. For example, distal portion 1104b may include a bone interface (e.g., one or more rigid struts) to promote mechanical coupling to the bone structure, and proximal portion 1104a may provide a grid (e.g., a rigid web structure) for strengthening/reinforcing the layer of filler (e.g., a bone void filler, cement, or bone graft material) provided within the gap between the implant and the bone interface.

In some embodiments, a height (h) of structure 1104 may be varied to provide for varying overall heights (H) of implant 1100. For example a first implant may have a structure 1104 having a height (h) of about two inches (e.g., about 5.08 cm) coupled to an implant body having a height of about one inch (e.g., about 2.54 cm) to give an overall implant height (H) of about two inches (e.g., about 5.08 cm). A proximal portion 1104a of the structure of first implant may be about one inch (e.g., about 2.54 cm) such that about two inches (e.g., about 5.08 cm) of the implant protrudes from the boney structure and about one inch of one inch (e.g., about 2.54 cm) of a distal portion 1104b of the structure is disposed into the boney structure the when the first implant is implanted. A second implant may have a structure 1104 having a height (h) of about three inches (e.g., about 7.62 cm) coupled to an implant body having a height of about one inch (e.g., about 2.54 cm) to give an overall implant height (H) of about four inches (e.g., about 10.16 cm). A proximal portion 1104a of the structure of first implant may be about two inches (e.g., about 5.08 cm) such that about two inches (e.g., about 5.08 cm) of the implant protrudes from the boney structure and about one inch of one inch (e.g., about 2.54 cm) of a distal portion 1104b of the structure is disposed into the boney structure the when the first implant is implanted. Where a patient has had about two-inches (e.g., about 5.08 cm) of bone removed, the first implant, having about two-inches (e.g., about 5.08 cm) protruding after being implanted, may be selected/used to compensate for the amount of bone removed. Where a patient has had about three-inches (e.g., about 7.62 cm) of bone removed, the second implant, having about three-inches (e.g., about 7.62 cm) protruding after being implanted, may be selected/used to compensate for the amount of bone removed. In some embodiments, a medical practitioner (e.g., a surgeon) may be provided a kit including a plurality of implants having different structure heights (h) and/or implant heights (H), and may select an appropriately sized/height implant for the particular application.

In some embodiments, stops may be provided to inhibit insertion (e.g., over insertion) of proximal portion 1104a into the bone structure. For example, stops may be provided at or near the transition between proximal and distal portions 1104a and 1104b (e.g., as illustrated by dashed line 1107). Exemplary stops are described in more detail below with respect to FIGS. 18A-18C. Such configurations may facilitate a gap to be formed between surface 1106 and a surface of adjacent bone structure when implant 1100 is pressed into the bone structure. For example, stops located at or near transition 1107 may completely or at least substantially inhibit proximal portion 1104a from being pressed into the bone structure. The resulting gap may be spanned by proximal portion 1104a. The resulting gap and/or voids within proximal portion 1104a may be impregnated or otherwise filled with filler (e.g., a bone void filler, cement, or bone graft material) that provides for adhering/coupling of implant 1100 to the bone structure.

FIG. 16 is a block diagram that illustrates implant 1100 implanted in accordance with one or more embodiments of the present technique. In the illustrated embodiment, distal portion 1104b is implanted into receiving bone structure 1120 such that transition 1107 between proximal and distal portions 1104a and 1104b is approximately even with a surface 1122 of bone structure 1120. Surface 122 may be formed via removal of bone to provide a suitable location for installation of implant 1100. For example, in the context of a knee implant a surface of bone structure 1120 may be shaved-off to provide a surface for the receipt of implant 1100. Bone structure 1120 may be similar to that of bone structure 120 described above. As a result of installing implant 110, gap 1108 may formed between surface 1122 of bone structure 1120 and surface 1106 of body 1102 of implant 1100. Gap 1108 may be spanned by proximal portion 1104a of structure 1104. Gap 1108 may be may be impregnated or otherwise filled with filler (e.g., a bone void filler, cement, or bone graft material) that provides for adhering/coupling of implant 1100 to bone structure 1120.

The bonding agent may be provided between members (e.g., struts) of interface structure 1104. In some embodiments, the bonding agent is provided in some or all of the proximal and/or distal portions 1104a and 1104b of interface structure 1104. For example, filler (e.g., a bone void filler, cement, or bone graft material) 1105 may be injected within distal portion 1104b and/or proximal portion 1104a, prior to insertion of implant 1100. During implantation, filler 1105 may be pushed into the proximal portion 1104a as distal portion 1104b is advanced into the bone structure, thereby filling some or all of gap 1108 and/or filler 1105 may penetrate into bone structure 1120, thereby facilitating coupling of at least distal portion 1104b to the bone structure. In some embodiments, a filler is injected into the resulting gap 108 during or after insertion of implant 1100. For example, after some or all of distal portion 1104b is advanced into bone structure 1120, filler 1105 may be injected into gap 108 (e.g., via an injection port as described below).

Interface structure 1104 may include a variety of open structures that may provide for insertion into the boney surface and/or provide for an open structure disposed within the resulting gap that is capable of receiving a bonding agent disposed therein. For example, proximal and/or distal portions 1104a and/or 1104b may include any of the strut/truss/web structures described and referred to herein.

In some embodiments, interface structure 1104 may include a proximal portion 1104a that extends a first distance from surface 1106 of body 1102 and a distal portion 1104b that extends a second distance that from surface 1106 of body 1102, where the second distance is greater than the first distance. In some embodiments, interface structure 1104 may include distal portion 1104b extending from (or otherwise integral with) proximal portion 1104a. For example, interface structure 1104 may include distal portion 1104b and proximal portion 1104a, as discussed below with regard to at least FIG. 17. In some embodiments, interface structure 1104 may include one or more structures that extend from surface 1106 of body 1102. For example, as depicted in FIG. 10a, implant 300 may includes a proximal rod structure 308a (e.g., a proximal portion) extending a first distance from contact face 306 of body 302 of implant 300 and a distal rod structure 308b (e.g., a distal portion) extending a second distance from contact face 306 of body 302 of implant 300, where the second distance is greater than the first. In some embodiments, proximal and distal portions may be separate structures from one another. For example, proximal rod structure 308a may be a separate structure from distal rod structure 308b. In some embodiments, proximal and distal portions may be coupled to and/or extend from a surface of the body of the implant. For example, proximal rod structure 308a and distal rod structure 308b may extend from contact face 306 of body 302 of implant 300. In some embodiments, proximal and distal portions may be directly coupled to (or otherwise extend directly from) a surface of the body of the implant. For example, proximal rod structure 308a may be directly coupled to contact face 306 of body 302 of implant 300 and distal rod structure 308b may be directly coupled to contact face 306 of body 302 of implant 300.

FIG. 17 is a block diagram that illustrates implant 1100 including a truss/web interface structure 1130 in accordance with one or more embodiments of the present technique. In the illustrated embodiment, truss/web interface structure includes a multi-layer rod-structure (e.g., truss/web structure), similar to that of multi-layer rod-structure (e.g., truss/web structure) 270 described with respect to FIG. 6. Each of proximal and distal portions 1104a and 1104b include four layers of rod-structures disposed one-a-top the other. In the illustrated embodiment, distal portion 1104a includes a web-structure coupled to and extending from a web structure of the proximal portion 1104a. Interface structure 1104 may include a proximal portion 1104a that extends a first distance from surface 1106 of body 1102 and a distal portion 1104b that extends a second distance from surface 1106 of body 1102, where the second distance is greater than the first distance.

In the illustrated embodiment, body 1102 includes an injection port 1130 extending there through. Injection port 1130 includes a conduit that extends from an access port 1130a to a plurality of injection outlets 1130b that terminate through surface 1106. Outlets 1130b may be distributed across surface 1106 to promote an even distribution of substances injected via pot 1130 into structure 1104. During use, a filler (e.g., a bonding agent) may be injected into proximal portion 1104a and/or distal portion 1104b via injection port 1130. For example, prior to, during, or after insertion of distal portion 1104b into bone structure 1120, a filler source (e.g., tube of cement) may be coupled to inlet 1130a and filler 1105 may be injected into injection port 1130 such that it exits outlets 1130b and is disposed within interior voids/openings of at least proximal portion 1104a.

FIG. 18A-18C are diagrams that illustrate stops 1370, 1380 and 1390 in accordance with one or more embodiments of the present technique. As described above, stops may be provided to inhibit insertion of proximal portion 1104a into the bone structure. For example, stops may be provided at or near transition 1107 between proximal and distal portions 1104a and 1104b. Disposing a stop at or near the transition between the proximal and distal portions of the interface structure may facilitate insertion of the distal portion while inhibiting insertion of the proximal portion into the bone structure.

In some embodiments, a stop may include one or more struts/members of an increased diameter/width relative to struts/members of distal portion 1104b. FIG. 18A illustrates a stop 1370 that includes three thick struts 1370a. Thick struts 1370 may have a cross-section that is the same, substantially the same or larger than that of adjacent struts of proximal and/or distal portions 1304a and 1304b. For example, struts 1370 may have a cross-sectional area, width and/or diameter that is about 105%, 110%, 115%, 120%, 125%, 140%, 150%, 175%, 200% of that of other struts (e.g., struts 1304a and/or 1304b). The thinner strut of distal portion 1104b may be readily inserted into the bone structure, whereas the thicker strut 1370a of the stop may not easily penetrate the bone structure. In some embodiments, struts 1370a may be oriented substantially parallel to surface(s) 1106 and/or 1122.

In some embodiments, a stop may include one or more planar members located at or near interface 1107. FIG. 18B illustrates a stop 1380 that includes a planar surface extending between struts of bone interface structure 1304. Surface of stop 1380 may be solid or substantially solid. For example, stop 1380 may include solid or substantially solid (e.g., perforated) plate that occupies about 10%, 20%, 30%, 40%, 50%, 60%, 70%, 80%, 90% or 100% of the area spanned between the struts by stop 1380. FIG. 18C illustrates a plurality of stops 1390 that each include a planar surface coupled to struts of bone interface structure 1304. The thinner strut of distal portion 1104b may be readily inserted into the bone structure, whereas the plate shaped stop 1380 may not easily penetrate the bone structure. In some embodiments, stops 1380 and/or 1390 may be oriented substantially parallel to surface(s) 1106 and/or 1122 and/or interface 1107. Surface of stops 1390 may be solid or substantially solid. For example, stop 1390 may include a solid or substantially solid (e.g., perforated) plate. Stop 1390 may have a cross-sectional area, width and/or diameter that is about 105%, 110%, 115%, 120%, 125%, 140%, 150%, 175%, 200%, 300%, 400% or of that of the cross-sectional area of the strut it is coupled to, (e.g., struts 1304a and/or 1304b).

FIG. 19 is a flowchart that illustrates a method 1400 of implanting an implant in accordance with one or more embodiments of the present technique. Method 1400 generally selecting an implant, preparing a bone structure, providing a bonding agent, installing the implant, and allowing the bonding agent to cure. Method 1400 may be employed for implanting implant 1100.

Method 1400 may include selecting an implant, as depicted at block 1401. Selecting an implant may include selecting an appropriately sized implant from one or more implants (e.g., an implant kit). In some embodiments, a medical practitioner (e.g., a surgeon) may select an implant from a kit including a plurality of implants having different structure heights and/or implant heights, and may select an appropriately sized/height implant for the particular application. For example, where a patient has had about two-inches (e.g., about 5.08 cm) of bone removed, an implant having a height of about two-inches (e.g., about 5.08 cm) may be selected/used to compensate for the amount of bone removed. Where a patient has had about three-inches (e.g., about 7.62 cm) of bone removed, an implant having about three-inches (e.g., about 7.62 cm) protruding after being implanted (e.g., the second implant described above) may be selected/used to compensate for the amount of bone removed.

Method 1400 may include preparing a bone structure, as depicted at block 1402. Preparing a bone structure may including removing a portion of the boney structure. In the case of a knee implant, for example, weight-bearing surfaces of the knee joint may be removed (e.g., shaved). In some embodiments, preparing a bone structure includes cutting/slitting the bone structure to accommodate one or more struts of the distal portion 1104b of implant 1100. For example, a cutting member may be advanced into the bone structure to create one or more cuts/slits that can accommodate struts of a distal bone interface portion 1104b.

Method 1400 may include providing a bonding agent, as depicted at block 1404. Providing a bonding agent may include providing a filler (e.g., a bone void filler, cement, or bone graft material) that is to be disposed within gap 1108 when implant 1100 is installed. For example, cement may be injected or otherwise provided within proximal and/or distal bone interface portions 1104a and 1104b. In some embodiments, filler is simply spread into bone interface structure 1104. In some embodiments, filler is injected into bone interface structure 1104 via injection port 1130. A filler may include a bone growth facilitator to provide or otherwise encourage the growth of bone into gap 1108 and/or proximate portions of distal portion 1104b disposed within bone structure 1120.

Method 1400 may include installing the implant, as depicted at block 1406. Installing the implant may include pressing implant 1100 into the prepared bone structure. For example, distal bone interface portion 1104b may be pressed into bone structure 1120. In some embodiments, implant 1100 is advanced until interface 1107 between distal and proximal bone interface portions 1104a and 1104b is at or near bone surface 1122. In some embodiments, implant 1100 is advanced until one or more stops (e.g., stop 1370, stop 1380, stop 1390) engage bone surface 1122 and/or inhibit further advancement of implant 1100.

In some embodiments, structure 1104 of implant 1100 may not be disposed substantially within bone structure 1120. For example a lower/outer edge portion of structure 1104 may simply abut surface 1122 of bone structure 1120. For example, a distal portion of structure 1104 may not be pressed into bone structure 1120 at all, or at least may not be pressed into bone structure 1104 with a substantial force, such that a distal portion of structure 1104 merely abuts bone structure 1120, or at least does not extend into bone structure 1120 a substantial distance. During use, structure 1104 may provide structural reinforcement for the gap between surface 1122 of bone structure 1120 and interface surface 1106 of implant 1100. In such an embodiment, installing implant (block 1406) may include abutting structure 1120 to surface 1122 of the bone. A filler may be provided within gap, as described above.

Method 1400 may include allowing the bonding agent to cure, as depicted at block 1408. In some embodiments, allowing the bonding agent to cure include allowing the cement to harden to provide sufficient bond between implant 1100 and bone structure 1120.

As will be appreciated, method 1400 is exemplary and is not intended to be limiting. One or more of the elements described may be performed concurrently, in a different order than shown, or may be omitted entirely. Method 1400 may include any number of variations. For example, in some embodiments, a bonding agent may be provided during and/or after installation of implant 1406. Accordingly, in some embodiments, steps 1404 and 1406 of method 1000 may be reversed or combined into a single step.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Furthermore, note that the word “may” is used throughout this application in a permissive sense (i.e., having the potential to, being able to), not a mandatory sense (i.e., must). The term “include”, and derivations thereof, mean “including, but not limited to”. As used throughout this application, the singular forms “a”, “an” and “the” include plural referents unless the content clearly indicates otherwise. Thus, for example, reference to “a member” includes a combination of two or more members. The term “coupled” means “directly or indirectly connected”.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

1. An orthopedic implant, comprising:
an implant body comprising a bone interface surface having a bone interface structure protruding therefrom, wherein the bone interface structure comprises:
a proximal portion of the bone interface structure adjacent the bone interface surface; and
a distal portion of the bone interface structure extending from the proximal portion of the bone interface structure, wherein the distal portion of the bone interface structure configured to be disposed at least partially into a bone structure during use.

2. The orthopedic implant of claim 1, wherein the proximal portion of the bone interface structure comprises one or more openings configured to receive a bonding agent within the one or more openings during use.

3. The orthopedic implant of claim 1, wherein the proximal portion of the bone interface structure comprises a web structure comprising a plurality of struts connected at nodes.

4. The orthopedic implant of claim 1, wherein the distal portion of the bone interface structure comprises a web structure comprising a plurality of struts connected at nodes.

5. The orthopedic implant of claim 1, wherein the distal portion of the bone interface structure comprises rod structure.

6. The orthopedic implant of claim 1, wherein the distal portion of the bone interface structure comprises at least one of a V-shaped structure, a U-shaped structure, and a hook-shaped structure.

7. The orthopedic implant of claim 1, wherein the bone interface structure comprises one or more stops configured to inhibit insertion of the proximal portion of the bone interface structure into the bone structure.

8. The orthopedic implant of claim 1, wherein the distal portion of the bone interface structure is configured to be pressed into the bone structure such that the distal portion bone interface structure at least partially penetrates the bone structure.

9. The orthopedic implant of claim 1, wherein the implant body comprises an injection port comprising a conduit configured to route a bonding agent into the bone interface structure.

10. The orthopedic implant of claim 1, wherein one or more portions of the bone interface structure comprise a biologic, growth factor or pain medication disposed thereon.

11. The orthopedic implant of claim 1, wherein the orthopedic implant comprises one or more of a large joint implant, a small joint implant, a trauma implant, a spine implant, a cranial maxi facial implant, a dental implant a knee implant, an ankle implant, a foot implant and a shoulder implant.

12. The orthopedic implant of claim 1, wherein the distal portion of the bone interface structure is configured to inhibit uplift of the orthopedic implant during use.

13. The orthopedic implant of claim 1, wherein the distal portion of the bone interface structure is configured to inhibit migration of the orthopedic implant during use.

14. The orthopedic implant of claim 1, wherein the distal portion of the bone interface structure is configured to inhibit rotation of the orthopedic implant during use.

15. An implant, comprising:
an implant body; and
a bone interface structure, comprising:
a distal bone interface structure; and
a proximal bone interface structure located between the distal bone interface structure and the implant body,
wherein the distal portion of the bone interface structure configured to be disposed at least partially into a bone structure during use, and
wherein the proximal portion of the bone interface structure is configured to be disposed in a gap between the bone structure and the implant body during use.

16. A method for providing an orthopedic implant, comprising:
inserting, into a bone structure, a distal portion of a bone interface structure coupled to an implant body such that a gap is formed between the implant body and the bone structure, and wherein the gap is spanned by a proximal portion of the bone interface structure extending between the implant body and the distal portion of the bone interface structure; and
providing a bonding agent within the gap.

17-34. (canceled)

35. The orthopedic implant of claim 3′1 claim 1, wherein the distal portion comprises a structure extending from the proximal portion.

36. The orthopedic implant of claim 3′1 claim 1, wherein the proximal portion comprises a first structure directly coupled to the bone interface surface of the implant and the distal portion comprises a second structure directly coupled to the bone interface surface.

37-49. (canceled)

50. The orthopedic implant of claim 1, wherein the proximal portion of the bone interface structure extends a first distance from the bone interface surface; and the distal portion of the bone interface structure extends a second distance from the bone interface surface, wherein the second distance is greater than the first distance.
